(19) United States
(12) Patent Application Publication
(10) Pub. No.: US 2011/0257458 A1
(43) Pub. Date: Oct. 20, 2011

(54) METHOD OF USING TUMOR CELL DEBRIS TO REDUCE BRAIN TUMOR RECURRENCE OR GROWTH

(75) Inventors: Dwain Morris-Irvin, Los Angeles, CA (US); Keith L. Black, Los Angeles, CA (US)

(73) Assignee: CEDARS-SINAI MEDICAL CENTER, Los Angeles, CA (US)

(21) Appl. No.: 13/141,193

(22) PCT Filed: Dec. 23, 2009
(86) PCT No.: PCT/US09/69437

§ 371 Date: Jun. 21, 2011

Related U.S. Application Data
(60) Provisional application No. 61/140,787, filed on Dec. 24, 2008.
(60) Provisional application No. 61/145,492, filed on Jan. 16, 2009.

(51) Int. Cl. A61M 37/00; A61K 39/39; A61B 18/18; A61P 35/00; A61M 5/32; A61K 39/00; A61P 37/04

(52) U.S. Cl. 600/1; 424/277.1; 424/257.1; 424/258.1; 424/278.1; 604/239; 606/33

(57) ABSTRACT

The present invention describes compositions comprising tumor cell debris, tumor lysate or tumor antigens and methods of using tumor cell debris, tumor lysate or tumor antigens for stimulating a specific immune response. The compositions may further comprise at least one TLR ligand. The stimulation of the specific immune response may be used to treat tumors, particularly, brain tumors. The treatment method may also reduce the recurrence of a tumor and/or inhibit or slow down the growth of a tumor.

### FIELD OF INVENTION

This invention relates to cancer treatment; particularly, treatment for brain tumors.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Cancer remains among the leading causes of death in the United States and around the world. Various forms of cancer are differentially treated, depending in part on the location of a tumor. One particularly difficult group of tumors to treat includes those that reside in and near the brain. Treatment of brain tumors presents a number of problems, not the least of which being the dangers inherent in any surgical procedure involving regions of the brain and the tissue located nearby. There is little room for error and the consequences of even a minor surgical mishap can be devastating to a patient; brain damage, or even death may result. Still, where possible, surgery remains a preferred method of treatment for most brain tumors and is often performed in conjunction with radiation therapy and chemotherapy. However, the infiltrative nature of this type of cancer makes complete surgical removal impossible. Even commonly referenced medical authorities suggest that patients with brain tumors be referred to centers specializing in investigative therapies, which is an indication that conventional modes of treatment are not overwhelmingly successful.

Glioblastoma multiforme, oligodendrogliomas, ependymomas and anaplastic astrocytomas are classified in the category of brain tumors commonly known as malignant gliomas. Gliomas are a type of brain tumor that account for 80% of malignant brain tumor diagnoses. Although relatively rare, gliomas are among the deadliest of tumors. Current treatments commonly include surgery followed by radiotherapy and chemotherapy.

One promising treatment method is the use of dendritic cell (“DC”) immunotherapy. DCs are antigen-presenting cells that naturally activate the patient's immune response by presenting antigens to T cells. In DC immunotherapy, the patient's dendritic cells are isolated, transfected with a tumor antigen, then injected back into the patient. When DCs expressing the tumor antigens are introduced into the patient, they increase the rate and extent of the patient's immune response against the tumor. However, DC immunotherapy requires dendritic cell isolation and transfection. Thus, there still exists a significant need in the art for additional treatment approaches; particularly, approaches that utilize the patient's own immune system.

### SUMMARY OF THE INVENTION

The following embodiments and aspects thereof are described and illustrated in conjunction with compositions and methods which are meant to be exemplary and illustrative, not limiting in scope.

Embodiments of present invention provide a method to elicit a specific immune response in a mammal in need thereof, comprising: providing a composition comprising a substance selected from the group consisting of tumor cell debris, tumor lysate, tumor antigens and combinations thereof, wherein the substance is generated by a method comprising exposing tumor cells to microwave radiation to induce cell death; and administering the composition to elicit a specific immune response in a mammal in need thereof. In one embodiment, the substance is tumor cell debris.

In another embodiment, the method may further comprise administering a composition comprising at least one toll-like receptor (TLR) ligand. In various embodiments, the at least one TLR ligand may be selected from the group consisting of TLR2 ligand, TLR4 ligand, TLR9 ligand and combinations thereof. In various embodiments, the at least one TLR ligand may be selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, TLR9 Agonist: ODN, and combinations thereof. In another embodiment, the method may further comprise administering a composition comprising toll-like receptor (TLR) ligands Pam3cys, LPS and CpG ODN.

In other embodiments of the method, the composition further comprises at least one toll-like receptor (TLR) ligand. In various embodiments, the at least one TLR ligand may be selected from the group consisting of TLR2 ligand, TLR4 ligand, TLR9 ligand and combinations thereof. In various embodiments, the at least one TLR ligand may be selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, TLR9 Agonist: ODN, and combinations thereof. In another embodiment, the composition may further comprise toll-like receptor (TLR) ligands Pam3cys, lipopolysaccharide (“LPS”) and CpG ODN.

In various embodiments, the tumor cell debris, and/or tumor lysate, and/or tumor antigens may be from brain tumor cells. In certain embodiments, the brain tumor cells may be from a type of brain tumor selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma, meningioma, pituitary carcinoma, neuroblastoma, craniopharyngioma and combinations thereof.

In certain embodiments, the specific immune response elicited by the method reduces the likelihood of tumor recurrence, slows down the growth of the tumor, and/or increases the survival time of the mammal.

Other embodiments of the present invention also provide a composition, useful for eliciting a specific immune response, comprising: a quantity of a substance selected from the group consisting of tumor cell debris, tumor lysate, tumor antigens and combinations thereof, wherein the substance is generated by a method comprising exposing tumor cells to microwave radiation to induce cell death; and a pharmaceutically acceptable carrier. In one embodiment, the substance is tumor cell debris.

In particular embodiments, the composition may further comprise at least one toll-like receptor (TLR) ligand. In various embodiments, the at least one TLR ligand may be selected from the group consisting of TLR2 ligand, TLR4 ligand, TLR9 ligand and combinations thereof. In other embodiments, the at least one TLR ligand may be selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40. TLR9 Agonist: ODN, and combinations thereof. In still other embodiments, the composition may further comprise toll-like receptor (TLR) ligands Pam3cys, LPS and CpG ODN.

In certain embodiments, the tumor cell debris, and/or tumor lysate, and/or tumor antigens are brain tumor cells. In various embodiments, the brain tumor cells may be from a type of brain tumor selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma, meningioma, pituitary carcinoma, neuroblastoma, craniopharyngioma and combinations thereof.

Other embodiments of the present invention provide for a kit, comprising: a pharmaceutically acceptable excipient adapted for preparing a composition comprising a quantity of a substance selected from the group consisting of tumor cell debris, tumor lysate, tumor antigens and combinations thereof, wherein the substance is generated by a method comprising exposing tumor cells to microwave radiation to induce cell death, for administration to a mammal in need of treatment to reduce the likelihood of tumor recurrence; and instructions for using the pharmaceutically acceptable excipient to prepare the composition for administration to the mammal.

In various embodiments, the kit may further comprise the composition comprising the quantity of the substance selected from the group consisting of tumor cell debris, tumor lysate, tumor antigens and combinations thereof.

In various embodiments, the kit may further comprise at least one toll-like receptor (TLR) ligand.

In various embodiments, the kit may further comprise one or more items selected from the group consisting of microfuge tube to maintain cells for freeze-thaw lysate procedure, phosphate buffered saline for the cells, 0.9% saline buffer for the cells, sterile plastic disposable forceps to manipulate cells into a microfuge tube, sterile water, 21-28 m gauge needle, 2 ml syringe to dissociate cells and cellular debris, 5 ml syringe to dissociate cells and cellular debris, Bradford dye, spectrophotometer cuvette for protein measurements, a quantity of bovine serum albumin (BSA) for standardizing protein concentrations, and 2 ml microfuge tube to store a lysate sample at a known concentration.

Still other embodiments of the invention provide for a method, comprising: providing a microwave probe; contacting the microwave probe to a tumor to ablate the tumor and generate tumor cell debris or using the microwave probe to deliver microwave radiation to the tumor to generate tumor cell debris to elicit a specific immune response in a mammal in need thereof.

In various embodiments, the method may further comprise administering a composition comprising at least one toll-like receptor (TLR) ligand. In various embodiments, the at least one TLR ligand may be selected from the group consisting of TLR2 ligand, TLR4 ligand, TLR9 ligand and combinations thereof. In other embodiments, the at least one TLR ligand may be selected from the group consisting of Pam3cys, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod; TLR8 Agonist: ssRNA40, TLR9 Agonist: ODN, and combinations thereof.

In various embodiments, the method may further comprise administering a composition comprising toll-like receptor (TLR) ligands Pam3cys, lipopolysaccharide (“LPS”) and CpG ODN.

In various embodiments, the tumor may be a brain tumor. In particular embodiments the brain tumor may be selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma; meningioma, pituitary carcinoma, neuroblastoma, craniopharyngioma and combinations thereof.

In various embodiments, the specific immune response may reduce the likelihood of tumor recurrence, slow down the tumor growth, and/or increase the survival time of the mammal.

Other features and advantages of the invention wilt become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts FACS analysis against GL26 cell microwaved for: (A) 5 seconds; (B) 10 seconds; (C) 20 seconds; (D) 30 seconds; and (E) 60 seconds in accordance with an embodiment of the present invention. FL1—Annexin V identifies apoptotic cells; FL2—Propridium Iodide distinguishes viable cells from nonviable cells.

FIG. 2 depicts a Kaplan Meier Survival Curve of GL26 glioma tumor in mice with intra-striatal injection of GL26 cellular debris generated after microwave exposure or saline. All mice previously exposed to cellular debris or saline survived longer than controls. P value<0.05.

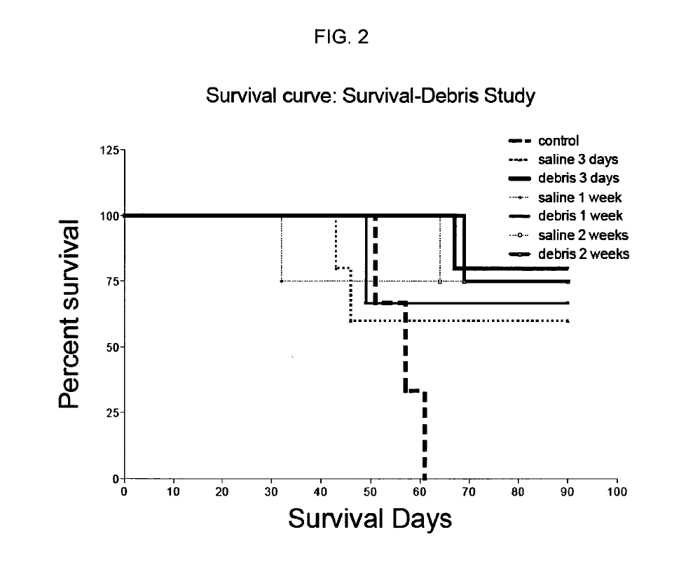

FIG. 3 depicts a Kaplan Meier Survival Curve of GL26 glioma tumor in mice with intra-striatal injection of CpG ODN or Pam3Cys, LPS and CpG together or saline 5 days after GL26 implantation. P value<0.05. These data suggest that intra-tumor injection of TLR ligands promote survival in glioma bearing mice.

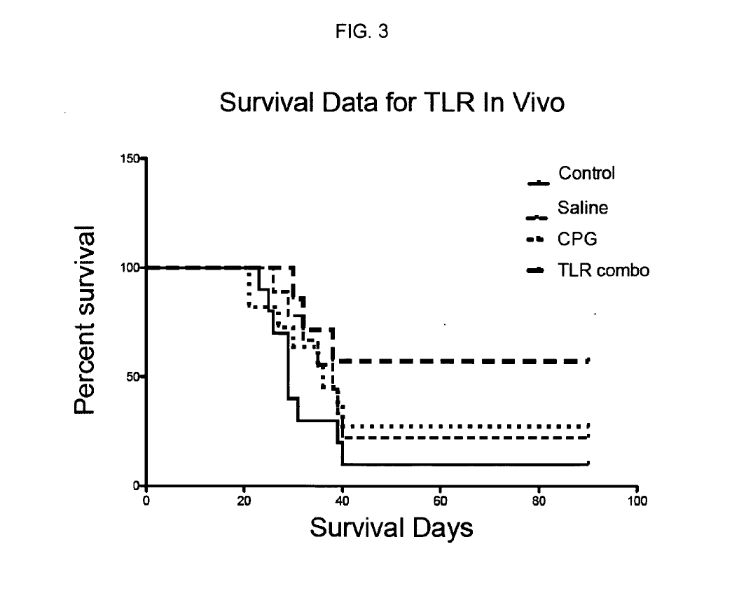

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be included within the scope of this term.

“Therapeutically effective amount” as used herein refers to that amount which is capable of achieving beneficial results in a patient with a tumor. A therapeutically effective amount can be determined on an individual basis and can be based, at least in part, on consideration of the physiological characteristics of the mammal, the type of delivery system or therapeutic technique used and the time of administration relative to the progression of the disease.

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary carcinomas, neuroblastomas, and craniopharyngiomas.

“Pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Treatment” and “treating,” as used herein refer to both therapeutic treatment and prophylactic or preventative measures, wherein the object is to prevent or slow down (lessen) the targeted pathologic condition or disorder even if the treatment is ultimately unsuccessful. Those in need of treatment include those already with the disorder as well as those prone to have the disorder or those in whom the disorder is to be prevented. For example, in tumor (e.g., cancer) treatment, a therapeutic agent may directly decrease the pathology of tumor cells, or render the tumor cells more susceptible to treatment by other therapeutic agents or by the subject's own immune system.

“Tumor,” as used herein refers to all neoplastic cell growth and proliferation, whether malignant or benign, and all pre-cancerous and cancerous cells and tissues.

In the present invention, the inventors have developed an immunotherapeutic treatment method for tumors, specifically, brain tumors, that enhances survival in an animal model. Rather than remove the patient's DC cells for modification and return, the inventors set out to stimulate the immune system in vivo. The inventors believe that the immune response may nonetheless be made tumor specific, as the subject's immune system will be stimulated with tumor cell debris and/or TLR ligands.

Using microwave radiation, the inventors selectively destroy tumor cells, and implant the cellular debris back into the brain of the subject, with or without the addition of Toll-like receptor (“TLR”) ligands. The data in a glioma mouse model showed that mice receiving the microwave-treated glioma cells showed a significant survival improvement over the control mice. A second control group that had saline implanted, without glioma cell debris, also showed improved survival rates. While not wishing to be bound by any particular theory, the inventors believe that the injury caused by the implantation may contribute to improved survival, through a host immune reaction. Injecting TLR ligands alone also served to enhance survival. In separate studies, combining microwaved-glioma implants along with TLR ligands enhanced the response and survival over either cellular debris or saline alone.

Embodiments of the present invention are based, in part, on the inventors' discovery that tumor cell debris, as well as TLR ligands can enhance survival. While not wishing to be bound by any particular theory, the inventors believe that glioma tumor cell debris generated after microwave exposure may be used as a “danger signal” to activate an antitumor immune response against the glioma. The inventors also believe that microwave ablation of the cells may release antigens, which stimulate the immune response, and these antigens may be different than antigens that are released in tumor lysate.

One embodiment of the present invention provides for a composition for eliciting a specific immune response (e.g., an anti-tumor response) in a mammal in need thereof. Particularly, the composition can be a vaccine useful for treating a brain tumor in a mammal in need thereof. For example, the composition may be used to reduce the recurrence of a tumor and/or inhibit or slow down the growth of a tumor. In one embodiment, the composition comprises tumor cell debris. In another embodiment, the composition comprises tumor lysate. In another embodiment, the composition comprises tumor antigens. In another embodiment, the composition further comprises at least one TLR ligand. Particularly useful TLR ligands include but are not limited to TLR2, TLR4 and TLR9 ligands. Examples of TLR ligands include but are not limited to Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, and TLR9 Agonist: ODN. One particularly useful embodiment is a combination of the following TLR ligands: Pam3cys, LPS and CpG ODN. In another embodiment, the composition comprises tumor cell debris, tumor lysate, and/or tumor antigens and at least one TLR ligand as described above.

Tumor cell debris may be generated by any method known in the art. In one embodiment, the tumor cell debris may be generated by exposing tumor cells to microwave radiation to induce cell death and cellular debris. In a particular embodiment, the tumor cell debris may be generated in situ using a microwave probe to deliver the microwave radiation to the tumor cells to induce cell death and cellular debris. For example, the practitioner may use the microwave probe to ablate the tumor in the brain and generate tumor cell debris in the brain. Thus, tumor resection may not be necessary.

Tumor lysate and tumor antigens may also be generated or provided any method known in the art.

In one particular embodiment, the tumor cell debris, tumor lysate or tumor antigens may be from a brain tumor, including but not limited to a glioma, glioblastoma, glioblastoma multiforme (GBM), oligodendroglioma, primitive neuroectodermal tumor, low, mid and high grade astrocytoma, ependymoma (e.g., myxopapillary ependymoma papillary ependymoma, subependymoma, anaplastic ependymoma), oligodendroglioma, medulloblastoma, meningioma, pituitary adenoma, pituitary carcinoma, neuroblastoma, and craniopharyngioma.

Another embodiment of the present invention is a method of eliciting a specific immune response (e.g., anti-tumor response) in a mammal in need thereof. The elicitation of the specific immune response may reduce the recurrence of a tumor and/or inhibit or slow down the growth of a tumor. The method comprises providing a composition comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above, and administering the composition to a mammal in need thereof to elicit the specific immune response. In another embodiment, the method comprises separately providing tumor cell debris, tumor lysate, or tumor antigens and the at least one TLR ligand and administering the tumor cell debris, tumor lysate, or tumor antigens (e.g., injection, intrastriatal) and the at least one TLR ligand (e.g., injection, intracranium) to a mammal in need thereof to elicit the specific immune response. The at least one TLR ligand can be administered with the tumor cell debris, tumor lysate, or tumor antigens, before the administration of tumor cell debris, tumor lysate, or tumor antigens, or after the administration of tumor cell debris, tumor lysate, or tumor antigens.

In another embodiment, the method of eliciting a specific immune response in a mammal in need thereof comprises providing a microwave probe; contacting the microwave probe to the tumor (and deliver microwave radiation) to ablate the tumor and generate tumor cell debris to elicit the specific immune response or using the microwave probe to deliver microwave radiation to the tumor cells to ablate the tumor and generate tumor cell debris to elicit the specific immune response. In one embodiment, contacting the microwave probe to the tumor or using the probe to deliver microwave radiation to the tumor is performed in situ. For example, a practitioner may contact the microwave probe to the tumor or use the probe to deliver microwave radiation to the tumor cells to ablate the tumor and generate tumor cell debris without resecting the tumor from the mammal's brain. In further embodiment, the method further comprises providing at least one TLR ligand and administering the at least one TLR ligand to a mammal in need thereof to elicit the specific immune response. The at least one TLR ligand can be administered substantially contemporaneous with the microwave ablation of the tumor, before the microwave ablation of the tumor or after the microwave ablation of the tumor.

In one embodiment, the method of eliciting a specific immune response is a method of treating cancer (e.g., a brain tumor). For example, the treatment method may reduce the recurrence of a tumor and/or inhibit or slow down the growth of a tumor. The method comprises providing a composition comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above, and administering the composition to a mammal in need thereof to treat the brain tumor. In another embodiment, the method comprises separately providing the tumor cell debris, tumor lysate, or tumor antigens, and the at least one TLR ligand and administering the tumor cell debris, tumor lysate, or tumor antigens and the at least one TLR ligand to a mammal in need thereof to treat the brain tumor. The at least one TLR ligand can be administered with the tumor cell debris, tumor lysate or tumor antigens, before the administration of tumor cell debris, tumor lysate or tumor antigens or after administration of the tumor cell debris, tumor lysate or tumor antigens.

In another embodiment, the method of treating cancer in a mammal in need thereof comprises providing a microwave probe; contacting the microwave probe to the tumor (and deliver microwave radiation) to ablate the tumor and generate tumor cell debris to elicit the specific immune response and treat cancer, or using the probe to deliver microwave radiation to the tumor cells to ablate the tumor and generate tumor cell debris to elicit the specific immune response and treat cancer. In one embodiment, contacting the microwave probe to the tumor or using the probe to deliver microwave radiation to the tumor is performed in situ. For example, a practitioner may contact the microwave probe to the tumor or use the probe to deliver microwave radiation to the tumor cells to ablate the tumor and generate tumor cell debris without resecting the tumor from the mammal's brain. In further embodiment, the method further comprises providing at least one TLR ligand and administering the at least one TLR ligand to a mammal in need thereof to treat cancer. The at least one TLR ligand can be administered substantially contemporaneous with the microwave ablation of the tumor, before the microwave ablation of the tumor or after the microwave ablation of the tumor.

Brain tumors treated by the inventive methods include but are not limited to gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, pituitary carcinomas, neuroblastomas, and craniopharyngiomas.

The compositions described above may be administered one or more times to the mammal to impart beneficial results. In various embodiments, the composition may be administered prior or post surgical resection of a tumor. For example, tumor cells obtained from a biopsy may be used to generate tumor cell debris, tumor lysate or tumor antigens for use in the compositions or methods of the present invention. Thus, in one embodiment, the composition may be administered prior to surgical resection of a tumor or administered without surgical resection of a tumor. In another example, the resected tumor may be used to generate tumor cell debris, tumor lysate or tumor antigens for use in the compositions or methods of the present invention. Thus, the composition may be administered post surgical resection of the tumor. One skilled in the art will be able to determine the appropriate timing for administering the composition. The timing of the first and/or subsequent dose(s) of the composition may depend on a variety of factors, including but not limited to a patient's health, stability, age, and weight. The composition may be administered at any appropriate time interval; for example, including but not limited to once per week, once every two weeks, once every three weeks, and once per month. In one embodiment, the composition may be administered indefinitely. In another embodiment, the composition may be administered three times in two week intervals. Appropriate dosage of the composition may also depend on a variety of factors, including but not limited to a patient's health, stability, age, and weight.

In various embodiments, the composition is administered by injection or implantation. In one embodiment, the inventive composition may be administered directly into or in close proximity to the tumor. In another embodiment, the inventive composition may be administered directly into or in close proximity to the site of the resected tumor.

In embodiments wherein the subject has a brain tumor, the composition may be administered into any location of the brain (e.g., striatum). In one embodiment, the composition may be administered into the opposite side of the brain from where the tumor resides or had resided. In another embodiment, the composition may be administered directly into or in close proximity to the brain tumor. In another embodiment, the composition may be administered directly into or in close proximity to the site of the resected tumor.

Additionally, administering the composition may be performed in conjunction with other therapeutic treatments; for example, chemotherapy and radiation. In one embodiment, the inventive composition is administered by injection (e.g., direct injection, intrastriatal, intracranium, intravenous, intraarterial, etc.).

Another embodiment of the present invention provides for a method of producing the composition or vaccine of the present invention. The method comprises providing tumor cells; and generating cellular debris from the tumor cells. In one embodiment, generating cellular debris comprises microwaving the tumor cells. In another embodiment, the method comprises providing tumor cells; and generating tumor lysate from the tumor cells. In another embodiment, the method comprises providing tumor cells; and generating or selecting tumor antigens from the tumor cells. In a further embodiment, the method further comprises adding at least one TLR ligand to the composition or vaccine. Particularly useful TLR ligands include but are not limited to TLR2, TLR4 and TLR9 ligands. Examples of TLR ligands include, but are not limited to Pam3cys, PolyI:C, LPS, ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, and TLR9 Agonist: ODN. One particularly useful embodiment is to add a TLR ligand cocktail comprising the TLR ligands Pam3cys, LPS and CpG ODN to the composition comprising the tumor cell debris.

In another embodiment, generating cellular debris comprising using a microwave probe to microwave the tumor cells in situ to generate the tumor debris.

In various embodiments, the present invention provides pharmaceutical compositions including a pharmaceutically acceptable excipient along with a therapeutically effective amount of the composition comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above. “Pharmaceutically acceptable excipient” means an excipient that is useful in preparing a pharmaceutical composition that is generally safe, non-toxic, and desirable, and includes excipients that are acceptable for veterinary use, as well as for human pharmaceutical use. Such excipients may be solid, liquid, semisolid, or in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, and parenteral. “Transdermal” administration may be accomplished using a topical cream or ointment or by means of a transdermal patch. “Parenteral” refers to a route of administration that is generally associated with injection, including intrastriatal, intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical preparations may be made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective amount of the compositions comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above can be as indicated to the skilled artisan by the in vitro responses or responses in animal models. The actual dosage can depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described. For example, dosages of Pam3Cys may be 50 μg/ml/1-9×10⁶ cells for 12-16 hours; dosages of LPS of may be 50 ng/ml/1-9×10⁶ cells for 12-16 hours; dosages of CPG ODN may be 5 μM/1−9×10⁶ cells for 12-16 hours.

The present invention is also directed to a kit to elicit a specific immune response in a mammal with cancer (e.g., brain tumor) or to treat cancer (e.g., brain tumor). The kit is useful for practicing the inventive method of eliciting an immune response in a mammal with cancer or a method of treating cancer. The kit is an assemblage of materials or components, including at least one of the inventive compositions. Thus, in some embodiments the kit contains a composition comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above.

The exact nature of the components configured in the inventive kit depends on its intended purpose. In one embodiment, the kit is configured particularly for the purpose of treating mammalian subjects. In a particular embodiment, the kit is configured particularly for the purpose of treating human subjects. In further embodiments, the kit is configured for veterinary applications, treating subjects such as, but not limited to, farm animals, domestic animals, and laboratory animals.

In other embodiments, the kit is configured for purposes of preparing and administrating the composition comprising tumor debris to the mammalian subject in need thereof. The kit may comprise one or more of the following: microfuge tubes (e.g, to maintain cells for freeze-thaw lysate procedure), phosphate buffered saline or 0.9% saline buffer for the cells, sterile plastic disposable forceps to manipulate cells into microfuge tubes, sterile water to lyse blood cells and remove from tumor sample, 21-28 m gauge needles and 2 or 5 ml syringes to dissociate cells and cellular debris, 5 mls of Bradford dye and spectrophotometer cuvettes for protein measurements, quantities of bovine serum albumin (BSA) for standardizing protein concentrations (e.g., 1 gram), and 2 ml microfuge tubes to store lysate samples at known concentrations.

Instructions for use may be included in the kit. “Instructions for use” typically include a tangible expression describing the technique to be employed in using the components of the kit to effect a desired outcome, such as to elicit a specific immune response in a mammal with cancer (e.g., brain tumor), to treat cancer (e.g., brain tumor), to prepare the composition comprising tumor cell debris, tumor lysate or tumor antigens for administering (e.g., by injection) into the mammal, or to prepare the composition comprising tumor cell debris, tumor lysate or tumor antigens and at least one TLR ligand as described above for administering (e.g., by injection) into the mammal. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. In one embodiment, the packaging materials employed in the kit are those customarily utilized in vaccination therapy.

As used herein, the term “package” refers to a suitable solid matrix or material such as glass, plastic, paper, foil, and the like, capable of holding the individual kit components. Thus, for example, a package can be a glass vial used to contain suitable quantities of an inventive composition comprising (1) tumor cell debris, (2) tumor lysate, (3) tumor antigens, (4) at least one TLR ligand as described above, (5) tumor cell debris and at least one TLR ligand as described above, or (6) tumor cell debris, tumor lysate, and/or tumor antigens and at least one TLR ligand as described above. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

GL26 glioma cells grown in vitro were exposed to microwaves in order to induce cell death and cellular debris. Panasonic Microwave, model number NN-S666BA, with an approximate frequency range between 3 and 30 GHz was used; almost all cells are ablated/destroyed within this range. The debris generated from these cells was unilaterally injected into the brains (striatum) of C57B|/6 mice at 3, 7 and 14 days before implanting 50,000 GL26 glioma cells into the contralateral striatum (the syngeneic rodent model for glioma). As controls, saline was delivered at the same time points 3, 7 and 14 days prior to GL26 implantation. A survival study was subsequently performed.

It was observed that GL26 tumor bearing mice that received either microwave induced cell debris or saline survived longer than mice that had no treatment prior to tumor implantation. These data suggest that cell debris and general damage to brain tissue during cell or saline injection prevents tumor formation or prolongs survival.

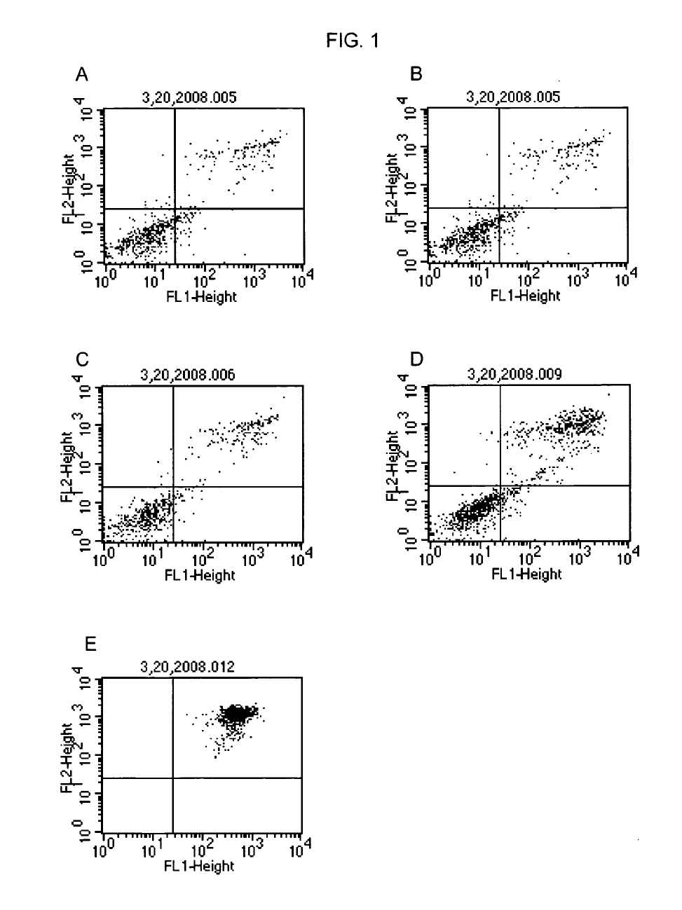

GL26 glioma cells were microwaved for 5 second, 10 second, 20 second, 30 second and 1 minute in standard DMEM culture medium. Subsequent cell apoptosis assay was performed by FACS analysis to determine cell death mechanism. (See FIG. 1.) After one minute of microwave exposure, all cells were double positive for both Annexin V and Propridium Iodide suggesting that all cells are dead.

Debris preparation: 50,000 of GL26 glioma cells were microwaved for 1 minute and viability was assessed by Trypan blue.

### Example 2

### Survival Experiment #1

Animals (Mouse strain: C57Bl/6) received saline or GL26 glioma cell debris on left site of the brain. One or two weeks later, 50,000 GL26 glioma cells were implanted into the contralateral striatum (right). The groups, with 5 animals in each group, were as follows: (1) Saline 1 week; (2) Debris 1 week; (3) Saline 2 weeks; and (4) Debris 2 weeks.

Date: 10/22 saline or debris; 10/29 GL26 glioma cells for 1 week; and 11/5 GL26 glioma cells for 2 weeks.

All animals receiving either saline or debris survived longer than animals that received no injection prior to 50,000 GL26 tumor cell implantation. These data suggest that injury and/or tumor debris promoted survival in experimentally-induced glioma bearing mice.

### Example 3

### Survival Experiment #2

Animals (C57/BL/6) received saline or GL26 glioma cell debris on the left side of the brain; 3 days, 1 week or 2 weeks later, 50,000 of GL26 glioma cells were implanted into the contralateral striatum (right).

The groups, with 5 animals in each group, were as follows: (1) Saline 3 days; (2) GL26 glioma cells 3 days; (3) Saline 1 week; (4) GL26 glioma cells 1 week; (5) Saline 2 weeks; and (6) GL26 glioma cells 2 weeks.

Date: 12/7 saline or GL26 glioma cell debris for 3 days or 2 weeks; 12/10 GL26 glioma cells for 3 days and control; 12/11 saline or GL26 glioma cells for 1 week; 12/18 GL26 glioma cells for 1 week; and 12/21 GL26 glioma cells for 2 weeks.

All animals receiving either saline or GL26 glioma cell debris survived longer than animals that received no injection prior to the 50,000 GL26 tumor cell implantation. These data suggest that injury and/or tumor debris promoted survival in experimentally-induced glioma bearing mice.

### Example 4

### Survival Experiment #3

50,000 GL26 glioma cells were implanted into the striatum (right) of the animals (C57/BL/6). 5 days later, TLR ligands CpG ODN alone or Pam3Cys, LPS and CpG ODN together were injected into the tumor.

TLR intracranial injection: LPS 5 μg+PAM3 10 μg+CPG 10 μg/2 μl were injected intracranially through the same location as the tumor was implanted on day 5 after tumor implantation.

CPG intracranial injection: CPG 10 μg/2 μl were injected intracranially through the same location tumor were implanted on day 5 after tumor implantation.

The groups were as follows: (1) GL26 alone; (2) GL26 with saline 5 days later; (3) GL26 with CpG ODN 5 days later; and (4) GL26 with Pam3Cys, LPS and CpG ODN 5 days later.

Animals receiving intra-tumoral injection of TLR ligands CpG ODN or Pam3Cys, LPS and CpG ODN together 5 days after tumor implantation survived longer than non-treated or saline injected mice. These data suggest intra-tumoral delivery of TLR ligands promotes survival.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method, comprising:
providing a composition comprising a substance selected from the group consisting of tumor cell debris, tumor lysate, tumor antigens and combinations thereof, wherein the substance is generated by a method comprising exposing tumor cells to microwave radiation to induce cell death; and
administering the composition to elicit a specific immune response in a mammal in need thereof.

2. The method of claim 1, wherein the substance is tumor cell debris.

3. The method of claim 1, further comprising administering a composition comprising at least one toll-like receptor (TLR) ligand.

4. The method of claim 3, wherein the at least one TLR ligand is selected from the group consisting of TLR2 ligand, TLR4 ligand, TLR9 ligand and combinations thereof.

5. The method of claim 3, wherein the at least one TLR ligand is selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, TLR9 Agonist: ODN, and combinations thereof.

6. The method of claim 1, further comprising administering a composition comprising toll-like receptor (TLR) ligands Pam3cys, lipopolysaccharide (“LPS”) and CpG ODN.

7. The method of claim 1, wherein the composition further comprises at least one toll-like receptor (TLR) ligand.

8. The method of claim 7, wherein the at least one TLR ligand is selected from the group consisting of TLR2 ligand, TLR4 ligand, TLR9 ligand and combinations thereof.

9. The method of claim 7, wherein the at least one TLR ligand is selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7Agonist: Imiquimod, TLR8 Agonist: ssRNA40, TLR9 Agonist: ODN, and combinations thereof.

10. The method of claim 1, wherein the composition further comprises toll-like receptor (TLR) ligands Pam3cys, lipopolysaccharide (“LPS”) and CpG ODN.

11. The method of claim 1, wherein the tumor cell debris, and/or tumor lysate, and/or tumor antigens are from brain tumor cells.

12. The method of claim 11, wherein the brain tumor cells are from a type of brain tumor selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma, meningioma, pituitary carcinoma, neuroblastoma, craniopharyngioma and combinations thereof.

13. The method of claim 1, wherein the specific immune response reduces the likelihood of tumor recurrence, slows down the growth of the tumor, and/or increases the survival time of the mammal.

14. A composition, comprising:
a quantity of a substance selected from the group consisting of tumor cell debris, tumor lysate, tumor antigens and combinations thereof, wherein the substance is generated by a method comprising exposing tumor cells to microwave radiation to induce cell death; and
a pharmaceutically acceptable carrier.

15. The composition of claim 14, wherein the substance is tumor cell debris.

16. The composition of claim 14, further comprising at least one toll-like receptor (TLR) ligand.

17. The composition of claim 16, wherein the at least one TLR ligand is selected from the group consisting of TLR2 ligand, TLR4 ligand, TLR9 ligand and combinations thereof.

18. The composition of claim 16, wherein the at least one TLR ligand is selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, TLR9 Agonist: ODN, and combinations thereof.

19. The composition of claim 14, further comprising toll-like receptor (TLR) ligands Pam3cys, LPS and CpG ODN.

20. The composition of claim 14, wherein the tumor cell debris, and/or tumor lysate, and/or tumor antigens are brain tumor cells.

21. The composition of claim 20, wherein the brain tumor cells are from a type of brain tumor selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma, meningioma, pituitary carcinoma, neuroblastoma, craniopharyngioma and combinations thereof.

22. A kit, comprising:
a pharmaceutically acceptable excipient adapted for preparing a composition comprising a quantity of a substance selected from the group consisting of tumor cell debris, tumor lysate, tumor antigens and combinations thereof, wherein the substance is generated by a method comprising exposing tumor cells to microwave radiation to induce cell death, for administration to a mammal in need of treatment to reduce the likelihood of tumor recurrence; and
instructions for using the pharmaceutically acceptable excipient to prepare the composition for administration to the mammal.

23. The kit of claim 22, further comprising the composition comprising the quantity of the substance selected from the group consisting of tumor cell debris, tumor lysate, tumor antigens and combinations thereof.

24. The kit of claim 22, further comprising at least one toll-like receptor (TLR) ligand.

25. The kit of claim 22 further comprising one or more items selected from the group consisting of microfuge tube to maintain cells for freeze-thaw lysate procedure, phosphate buffered saline for the cells, 0.9% saline buffer for the cells, sterile plastic disposable forceps to manipulate cells into a microfuge tube, sterile water, 21-28 m gauge needle, 2 ml syringe to dissociate cells and cellular debris, 5 ml syringe to dissociate cells and cellular debris, Bradford dye, spectrophotometer cuvette for protein measurements, quantity of bovine serum albumin (BSA) for standardizing protein concentrations, and 2 ml microfuge tube to store a lysate sample at a known concentration.

26. A method, comprising:
providing a microwave probe;
contacting the microwave probe to a tumor to ablate the tumor and generate tumor cell debris or using the microwave probe to deliver microwave radiation to the tumor to generate tumor cell debris to elicit a specific immune response in a mammal in need thereof.

27. The method of claim 26, further comprising administering a composition comprising at least one toll-like receptor (TLR) ligand.

28. The method of claim 27 wherein the at least one TLR ligand is selected from the group consisting of TLR2 ligand, TLR4 ligand, TLR9 ligand and combinations thereof.

29. The method of claim 27, wherein the at least one TLR ligand is selected from the group consisting of Pam3cys, PolyI:C, lipopolysaccharide (“LPS”), ST-FLA, Gardiquimod, CpG ODN, TLR1/2 Agonist: Pam3CSK4, TLR2 Agonist: HKLM, TLR3 Agonist: Poly(I:C), TLR4 Agonist: LPS E. coli, TLR5 Agonist: Flagellin S. typhimurium, TLR6/2 Agonist: FSL1, TLR7 Agonist: Imiquimod, TLR8 Agonist: ssRNA40, TLR9 Agonist: ODN, and combinations thereof.

30. The method of claim 26, further comprising administering a composition comprising toll-like receptor (TLR) ligands Pam3cys, lipopolysaccharide (“LPS”) and CpG ODN.

31. The method of claim 26 wherein the tumor is a brain tumor.

32. The method of claim 31, wherein the brain tumor is selected from the group consisting of glioma, glioblastomas, glioblastoma multiforme (GBM), oligodendroglioma, primitive neuroectodermal tumor, low, mid and high grade astrocytoma, ependymoma, oligodendroglioma, medulloblastoma, meningioma, pituitary carcinoma, neuroblastoma, craniopharyngioma and combinations thereof.

33. The method of claim 26, wherein the specific immune response reduces the likelihood of tumor recurrence, slows down the tumor growth, and/or increases the survival time of the mammal.
